(19) United States
(12) Patent Application Publication
(10) Pub. No.: US 2012/0301939 A1
(43) Pub. Date: Nov. 29, 2012

(54) METHODS OF TREATING BIOMASS

(76) Inventors: J. T. Harvey, Lakewood, CO (US); Andrew W. Flemming, Littleton, CO (US)

(21) Appl. No.: 13/113,633

(22) Filed: May 23, 2011

(51) Int. Cl. C12P 7/10

(52) U.S. Cl. 435/165

(57) ABSTRACT

A process for producing biofuel from biomass that includes free monosaccharides is provided. The process comprises the steps of mixing the biomass with a recycled hydrolysate for a sufficient time to elute a portion of the free monosaccharides from the biomass thereby forming a sugar enriched hydrolysate. Sugar enriched hydrolysate is then separated from the biomass and monosaccharides contained in the separated sugar enriched hydrolysate are fermented.

### FIELD

The present patent document relates to apparatuses and methods for treating biomass. More particularly, the present patent document relates to apparatuses and methods for treating biomass that includes free sugars.

### BACKGROUND

As countries around the world become more industrialized and the world economy continues to increase in size, the demand and price of oil has continued to rise accordingly. This steady rise in the demand and price of oil has been further escalated by political unrest in major oil producing countries and the limited supply of oil as a resource.

In addition to the scarcity and price of oil, many scientists have recognized the potential damage to the Earth's atmosphere that may be caused by the consumption of oil as a fuel due to the additional carbon that is added into the atmosphere as a result. As the world economy continues to grow in size, and with it the volume of oil consumed, the amount of additional carbon added into the atmosphere is similarly increased. Accordingly, a significant amount of research and development has gone into the advancement of alternative renewable energy and fuel sources.

One recognized way to provide a renewable alternative source of energy is by extracting sugars from biomass and converting them into a consumable fuel. During the growing process biomass removes carbon dioxide from the atmosphere, as a result, using biomass as a fuel source is considered carbon neutral for the environment. Furthermore, biomass does not suffer the same limited resource problem that oil does because unlike oil, which forms deep in the ground over many thousands of years, biomass can be quickly regenerated in the next growing season.

Numerous processes have been developed to facilitate the conversion of biomass into an alternative energy source such as liquid biofuel. These processes suffer from numerous drawbacks including the formation of deleterious byproducts and lack of efficiency, which affect the overall viability of the technology.

### SUMMARY OF THE INVENTION

In view of the foregoing, an object according to one aspect of the present patent document is to provide improved apparatuses and processes for treating biomass that includes free sugars. Preferably, the apparatuses and processes address or at least ameliorate one or more of the problems described above. To this end, a process for producing biofuel from biomass that includes free monosaccharides is provided. The process comprises the steps of mixing the biomass with a recycled hydrolysate for a sufficient time to elute a portion of the free monosaccharides from the biomass thereby forming a sugar enriched hydrolysate. Sugar enriched hydrolysate is then separated from the biomass. These newly recovered monosaccharides in the separated sugar enriched hydrolysate are then fermented. The processes may be used to produce biofuel from any biomass that includes free sugars and in particular, may be used to produce ethanol from bagasse.

In one embodiment, the biomass is mixed with the hydrolysate for a time sufficient to elute a majority of the free monosaccharides into the recycled hydrolysate. In yet another embodiment, the mixing occurs until 90% or more of the free sugars are eluted into the recycled hydrolysate.

In one embodiment of the process, an additional step of pretreating the biomass to produce hydrolysate and a biomass residue is performed after the separating step. In yet another embodiment of the process that includes the additional pretreating step, a further step of separating the hydrolysate from the biomass residue is performed. In a further embodiment, the hydrolysate from the separating step is used as the recycled hydrolysate in the mixing step for a new batch of biomass containing free monosaccharides.

In some embodiments, the separating steps may be performed by pressing. Pressing may be used for either separating step, or both separating steps.

In at least one embodiment of the process, the biomass is sugarcane bagasse. The bagasse may be provided by a sugar mill as the byproduct of the production of sugar from sugarcane.

In yet another embodiment, the biomass residue that results from the pretreatment step is converted into a high value product.

In another aspect of the embodiments disclosed herein, a process for producing biofuel from biomass including free sugars is provided. The process comprises the steps of mixing the biomass with a liquid solution until a substantial percentage of free sugars in the biomass are eluted into the liquid solution, wherein the biomass is mixed with the liquid solution prior to pretreating the biomass. Liquid solution including the free sugars is then separated from the biomass and then free sugars in the separated liquid solution are fermented. In one embodiment of the process the liquid solution is hydrolysate.

In yet another embodiment of the process, the biomass is sugarcane bagasse. In certain embodiments, the sugarcane bagasse may be provided by a sugar mill.

In another aspect of the various embodiments disclosed herein, a process for producing biofuel from biomass that includes free sugars is provided. The process comprises the steps of mixing the biomass with a hydrolysate prior to pretreating the biomass. Free sugars contained within the biomass are eluted by the hydrolysate. Hydrolysate including the eluted free sugars is then separated from the biomass. Once the hydrolysate including the eluted free sugars is separated, the free sugars contained within the hydrolysate may be fermented.

In a further embodiment, the step of pretreating the biomass to produce a mixture of hydrolysate and biomass residue is performed. In yet another embodiment, the hydrolysate is separated from the biomass residue and the hydrolysate is recycled to be mixed with a new batch of biomass.

In another embodiment, the fermentation step is performed with immobilized microbes.

In yet another embodiment, the biomass residue is combusted in a combustion chamber. In certain embodiments, the combustion of the biomass residue is used to help drive the plant. In some embodiments, particularly embodiments where the biomass residue is combusted, the concentration of slag forming constituents in the biomass residue may be substantially reduced.

### BRIEF DESCRIPTION OF THE DRAWINGS

FIG. 1 illustrates a process for creating a biofuel from biomass.

FIG. 2 illustrates one embodiment of a process for producing a biofuel from biomass that includes free sugars.

FIG. 3. illustrates the process of FIG. 2 used in conjunction with a process for converting sugarcane into sugar.

FIG. 4 illustrates one embodiment of a process for producing a biofuel from biomass that includes free sugars.

### DETAILED DESCRIPTION OF THE PREFERRED EMBODIMENTS

In the following description of the preferred embodiments, reference is made to the accompanying drawings that form a part hereof, and in which specific embodiments are shown by way of illustration. It is to be understood that other embodiments may be utilized and structural changes may be made without departing from the scope of the present patent document.

Consistent with its ordinary meaning as a renewable energy source, the term “biomass” is used herein to refer to living and recently dead biological material including carbohydrates, proteins and/or lipids that may be converted to fuel for industrial production. By way of non-limiting example, “biomass” refers to plant matter, including, but not limited to switchgrass, sugarcane, sugarcane bagasse, sugar beets, corn stover, corn cobs, alfalfa, Miscanthus, poplar, and aspen, biodegradable solid waste such as dead trees and branches, yard clippings, recycled paper, recycled cardboard, and wood chips, plant matter listed above or animal matter, and other biodegradable wastes.

Consistent with its ordinary biological meaning, the term biomass that includes “free sugars” is used herein to refer to a subset of biomass that includes monosaccharides such as pentoses or hexoses that are not bound up in cellulose or hemicellulose structures. By way of non-limiting example, types of biomass that include “free sugars” are: sugarcane, bagasse, sugar beets, fruit processing waste, wood, and grasses to name a few. Also included in the definition of the term biomass that includes “free sugars” are biomass sources that may not naturally have “free sugars” but are preprocessed, by for example pressing, to release monosaccharides from the cellulose or hemicellulose into “free sugars.”

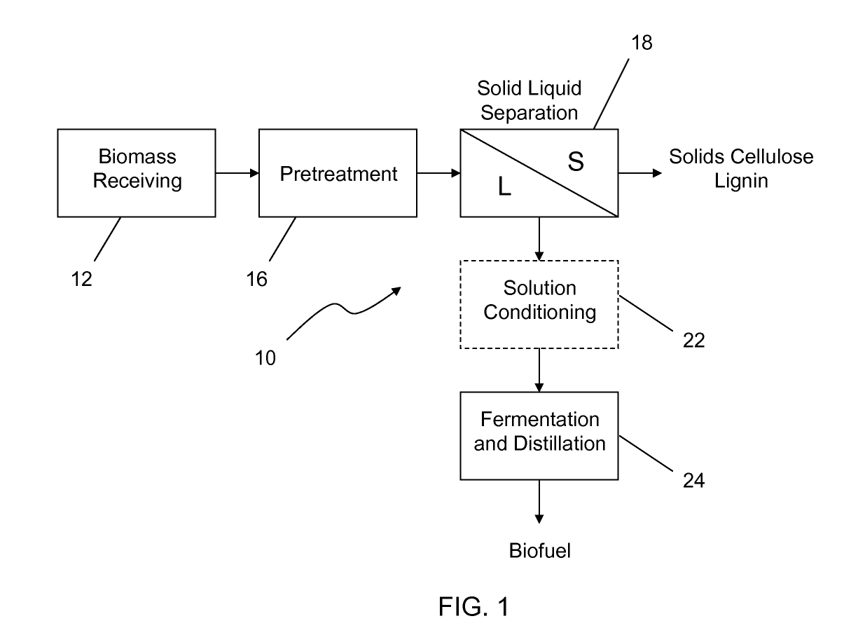

FIG. 1 illustrates a typical process 10 for creating biofuel from biomass. In process 10, biomass is received 12 and then pretreated 16. Prior to pretreatment 16, the biomass may be optionally preprocessed (not shown). One example of preprocessing is reducing the structural size of the biomass, such as for example chipping or chopping wood into smaller pieces to increase its susceptibility to pretreatment. The pretreated biomass then goes through a solid liquid separation 18. The liquid extracted from solid liquid separation 18 may then be conditioned 22 and fermented and distilled 24 into a biofuel.

The pretreatment step 16 is used to disrupt the polymer network of cellulose, hemicellulose, and lignin forming the biomass structure so the polysaccharides may be reduced to monosaccharides. This process is commonly referred to as “pretreatment” as shown in step 16 of FIG. 1. The pretreatment step 16 is designed to reduce the recalcitrance of the biomass to enzymatic or chemical saccharification. The pretreatment may reduce the recalcitrance of both the hemicellulose and cellulose in the biomass to enzymatic or chemical saccharification. In other embodiments, the pretreatment may reduce the recalcitrance of either the hemicellulose or cellulose or both in different varying amounts. Furthermore, in some embodiments the pretreatment may go further and also be responsible for saccharification of the hemicellulose and/or cellulose into their monosaccharide components. A liquid hydrolysate may be produced from bagasse or other biomass during pretreatment 16 using a number of methods including, for example, in a pressure reactor. Table 1 lists appropriate ranges for temperature, dwell time, and moisture content necessary for the separation and hydrolyzation of hemicellulose in a pressure reactor.

TABLE 1
Pressure Reactor Pretreatment Conditions

| Temperature* | 105-200° C. |
|---|---|
| Dwell time | 1 minute-24 hours |
| Moisture Content | 25-90% |

*Temperature dictates the pressure in a sealed vessel in a saturated steam environment

Reagents may be used to enhance the effectiveness of the pretreatment 16. Different biomass sources may respond better to the addition of different reagents. Reagents may include, but are not limited to: nitric acid, phosphoric acid, hydrochloric acid, sulphuric acid, sulphur dioxide, ethanol, sodium hydroxide and sodium sulphite. Other reagents that reduce the recalcitrance of the biomass to hemicellulose or cellulose removal may also be added.

In addition to performing the pretreatment step 16 in a pressure reactor, pretreatment step 16 may be performed using a number of other methods including acid prehydrolysis, steam cooking, alkaline processing, rotating augers, steam explosion, and ball milling. Pretreatment may not only liberate or extract the hemicellulose and cellulose but may also facilitate the breakdown of the hemicellulose and cellulose and solubilize the pentoses and hexoses at the same time to form a hydrolysate mixed with a biomass residue. Hydrolysing the pentoses and hexoses eliminates the need to add large amounts of enzymes to produce monomeric sugars in conditioning step 22.

Once hemicellulose and cellulose are liberated from the biomass and the monosaccarides are solubilized, fermentation 24 may begin. While fermentation 24 may occur within the hydrolysate and biomass residue mixture, preferably the hydrolysate containing the sugars is separated in step 18 via solid/liquid separation and/or washing the hydrolysate from the biomass residue and then fermented ex-situ in step 24. Depending on the biomass and treatments employed, the pretreatment and hydrolysis step 16 may yield soluble sugars from the biomass in the form of xylose, mannose, arabinose, galactose, and glucose ready for fermentation in step 24. Once sugars are fermented into a liquid biofuel they may be upgraded to a pure anhydrous fuel via conventional distillation and dehydration processes.

The present patent document teaches a method of increasing the effectiveness of the process 10 of FIG. 1 for biomass sources that already include free sugars present within them prior to pretreatment. Although pretreatment may be used to liberate free sugars held in the cellulose and hemicellulose, subjecting the free sugars that are present in certain biomass sources to pretreatment may damage the free sugars. Damaging the free sugars prevents them from being available for further processing into biofuel in the subsequent fermentation and distillation 24, thereby reducing the effectiveness and efficiency of the process 10.

In addition to the free sugars not being available for further processing into biofuel due to being damaged during pretreatment, exposing the free sugars to pretreatment may degrade them into inhibitory secondary products. The increase of inhibitory secondary products in the hydrolysate further hinders the ability of microbes to process the monosaccharides in the hydrolysate into biofuel in the fermentation and distillation step 24.

Inhibitory secondary products, which are inhibitory to the fermentation step 24, are produced or extracted from the biomass during pretreatment. In particular, free sugars may be degraded into inhibitory secondary products during pretreatment step 16. The concentrations of fermentation inhibitors that form in converting biomass to fermentable hexoses and pentoses will vary depending on the source of the biomass and the methods used for the pretreatment and hydrolysis step 16. For example acetic acid is produced by cleavage of acetyl groups from hemicellulose. Phenolic and polyphenolic compounds (collectively “Phenolic Compounds”) are also formed from the degradation of lignin. In addition, free pentoses and hexoses may be degraded due to dehydration into furfural and hydroxymethylfurfural (HMF). While the generated Phenolic Compounds, furfural, HMF, and acetic acid are all potentially valuable compounds, they are also fermentation inhibitors, and may prevent or inhibit fermentation, particularly as their concentrations increase.

In addition, furfural and HMF can further degrade to produce levulinic acid, acetic acid, and formic acid, which are even more potent fermentation inhibitors. Phenolic and polyphenolic compounds produced from hydrolysis of wood hemicellulose and the concomitant lignin degradation include guaiacol, vanillin, phenol, vanillic acid, syringic acid, salicylic acid, gentisic acid, and others. Many of these compounds, for instance vanillin and vanillic acid, are known to inhibit the growth of and/or fermentation with microbial yeasts, such as Pachysolen and Saccharomyces.

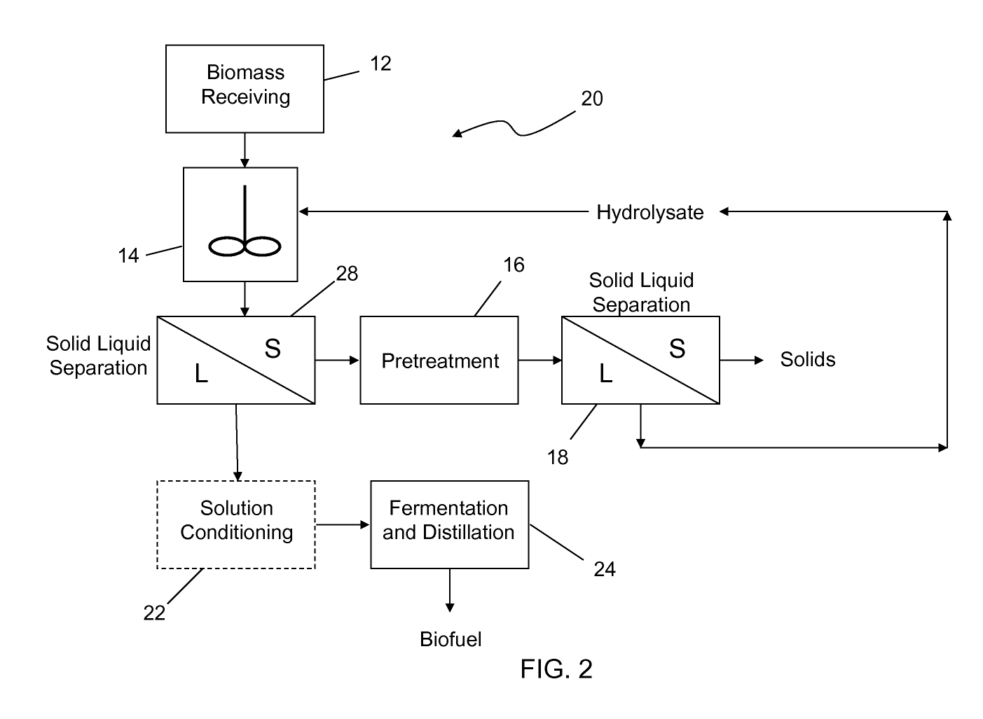

To this end, the present patent document teaches a new and novel method for processing biomass that includes free sugars (pentoses and hexoses) into biofuel such as ethanol. FIG. 2 illustrates one embodiment of a method 20 for processing biomass that includes free sugars into biofuel. In method 20, after the biomass is received 12, it is mixed with hydrolysate in mixing step 14. The hydrolysate may be recycled hydrolysate from a solid/liquid separation step downstream in the process or a recycled hydrolysate from another source. The hydrolysate and biomass solution are then further processed in a solid/liquid separation step 28. In solid/liquid separation step 28, the free sugars and hydrolysate are separated out from the biomass. Steps 14 and 28 occur prior to pretreatment 16 and are used to remove the free sugars from the biomass prior to pretreatment 16.

Removing the free sugars from the biomass prior to pretreatment 16, allows the free sugars to be processed into biofuel rather than pass through pretreatment and be degraded due to dehydration into inhibitory secondary products such as furfural and HMF. Accordingly, the benefits of process 20 are realized both in an increase of available sugars for processing into biofuel and in a reduction of inhibitory secondary products formed during pretreatment and present in the hydrolysate.

Referring to FIG. 1, the mixing step 14 is preferably performed until a majority of free sugars are eluted from the biomass into the hydrolysate to form a sugar enriched hydrolysate. More preferably a large percentage of the free sugars are eluted and even more preferably mixing step 14 is performed for a sufficient time to elute 90% or more of the free sugars into the hydrolysate to form a sugar enriched hydrolysate.

The solid/liquid separation step 28 separates the liquid, which includes hydrolysate and the free sugars eluted therein, from the biomass solid material. In a preferred embodiment, solid/liquid separation step 28, separates a majority of the free sugars from the biomass solids. More preferably, solid/liquid separation step 28 separates 75% or more of the free sugars from the biomass solids. Even more preferably, solid/liquid separation step 28 separates 85% or more of the free sugars from the biomass solids.

The biomass solid material from solid/liquid separation step 14 is pretreated 16 just as it would be in FIG. 1, except for now a majority of the free sugars have been removed. After pretreatment step 16, the material goes through an additional solid/liquid separation step 18. Similar to the solid/liquid separation step 18 in FIG. 1, the solid liquid step 18 in FIG. 2 separates the hydrolysate formed during pretreatment from the solid biomass residue. In the novel method of FIG. 2, the hydrolysate is recycled back into mixing step 14 rather than being passed directly on to conditioning 22 and fermentation and distillation 24.

In the process of FIG. 2, the solution conditioning 22 and fermentation and distillation 24 occur using the sugar enriched hydrolysate from solid/liquid separation step 28. The sugar enriched hydrolysate separated from solid/liquid separation step 28 includes not only the hydrolysate from solid/liquid separation step 18, but also includes a substantial amount of the free sugars that are eluted into the hydrolysate when it is mixed with the biomass in mixing step 14. The process 20 of FIG. 2 prevents a substantial portion of the free sugars that are naturally occurring in the biomass 12 from entering the pretreatment step 16.

The fermentation step 24 may be performed by any number of different microbial species including both yeasts and bacteria. The fermentation may occur in a batch process or a continuous process and may be placed in an appropriate bioreactor accordingly. Furthermore, the microbial species may be free or immobilized. Various processes and apparatuses for fermentation and immobilization are described in detail in United States Published Patent Applications No. 2011-0056126 and No. 2011-0059497 which are hereby incorporated by reference in their entirety. In addition, numerous processes and apparatuses for creating an encapsulated cell product are described in detail in U.S. patent application Ser. No. 13/027,267 which is hereby incorporated by reference in its entirety.

In at least one embodiment of the process 20, the biomass residue that remains after the hydrolysate is separated out in solid/liquid separation step 18 may continue on to be made into a high value product. For example, lignin and cellulose residue may be made into a high-energy density fuel for use in operating the plant or made into a paper mill feedstock.

Although in the embodiment shown in FIG. 2 the biomass that contains free sugars is mixed with hydrolysate in mixing step 14, other liquid solutions may be used instead of hydrolysate. Any liquid solution may be used that will elute the free sugars in the biomass. For example, in some instances it might be advantageous to recycle a wash water, condensate or specific downstream process solution as the eluting solution. Or in the case of a sugar mill, there may be internal streams in the existing operations that could be leveraged and integrated. As may be seen in FIG. 2, and in the preferred embodiment, hydrolysate is used in mixing step 14 because it is produced by the pretreatment step 16 and may be recycled into mixing step 14. Using hydrolysate also has other benefits including lowering the concentration of slag forming constituents in the biomass residue as will be discussed below.

When the process 20 is first initiated, hydrolysate may not be available for mixing step 14 because no biomass has passed through pretreatment step 16 yet. Consequently in some embodiments, process 20 may need to be bootstrapped with hydrolysate from a previous process. In other embodiments, process 20 may be initiated using an alternative liquid solution and then recycled hydrolysate is subsequently used once biomass begins to pass through the pretreatment step 16. In an alternative embodiment, a small batch of biomass is processed directly from receiving step 12 into pretreatment step 16 to produce hydrolysate to bootstrap mixing step 14 and process 20.

In addition to being a process designed specifically for creating biofuel such as ethanol, the process 20 illustrated in FIG. 2 may be inserted into or appended to a wider variety of existing commercial biomass processing plants, including for example, a sugarcane processing plant.

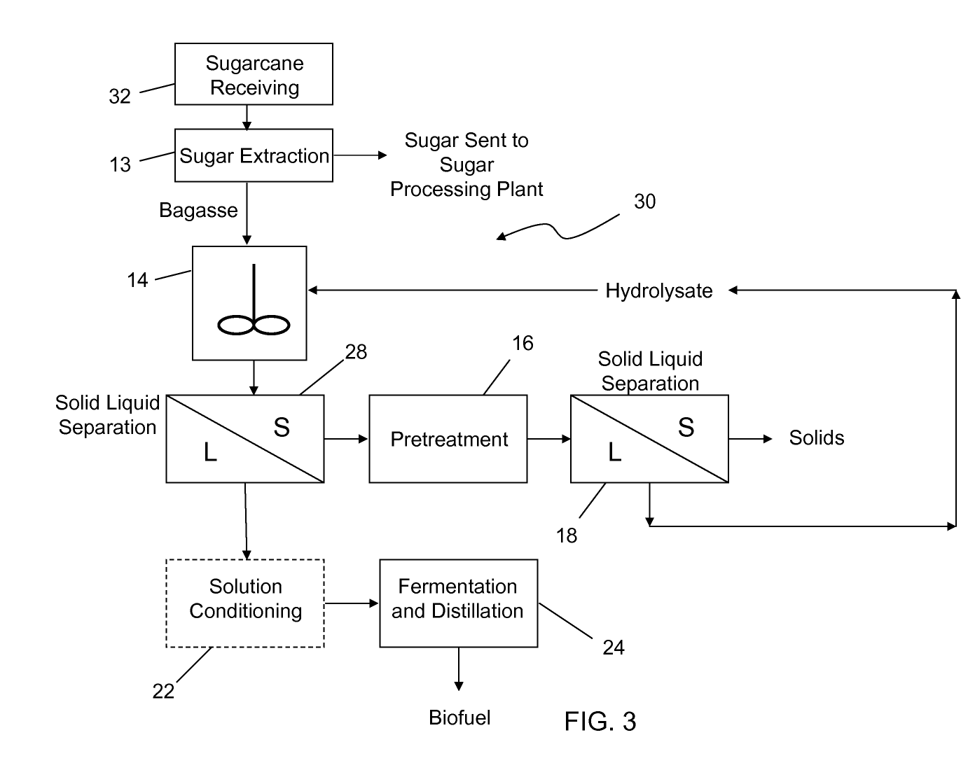

FIG. 3 illustrates the process of FIG. 2 used in conjunction with a sugar producing process. In the process 30 shown in FIG. 3, sugarcane is received 12 and processed as it normally would be by a sugar mill to extract the free sugar in sugar extraction step 13. Normal processes used by sugar mills to extract the free sugars in sugarcane do not extract 100% of the free sugars from the sugarcane. Consequently, free sugars still remain in the bagasse byproduct that is passed on for further processing into biofuel. In process 30 of FIG. 3, similar to process 20 in FIG. 2 discussed above, the free sugars are eluted by the hydrolysate in mixing step 14 and preserved for fermentation by being removed in solid/liquid separation step 28, rather than being passed onto pretreatment 16 where they would likely be degraded into inhibitory secondary products.

In the embodiments shown in FIGS. 2 and 3, the solid/liquid separation steps 18 and 28 may be performed using a number of methods including, but not limited to, centrifuging or pressing. The same method or different methods may be used for solid/liquid separation step 18 and solid/liquid separation step 28. If pressing is used, preferably pressing may be accomplished with a screw press. However, numerous other types of mechanical or machine presses may be used. For example, a press such as a hydraulic press, a hydro-mechanical press, a pneumatic press or any other type of press that can apply the necessary pressure to separate hydrolysate and free sugar solution from the solid biomass material or biomass residue may be used. The press may have a range of capabilities and configurations for pressing out the liquid. Preferably the press can generate from at least about 10.5 kg/cm² to about 21.1 kg/cm². In other embodiments, it is desirable if the press can generate at least approximately 1,410 kg/cm².

The final product that the biomass residue is to eventually be used for may determine what size and kind of press to use for solid/liquid separation step 18. For example, if the biomass residue is to eventually be used to generate cellulose fibers to make paper products, cardboard, or fiberboard, a lower pressure, such as in the range of 10.5 kg/cm² to 21.1 kg/cm² may be advantageous to minimize damage to the cellulose fibers. In processes that turn the biomass residue into high energy density fuel, higher pressures may be used to minimize the moisture content, without regard to fiber quality. As a result, it may be desirable to employ pressures of about 1,410 kg/cm² or even higher. In other embodiments, however, pressures within the range of 10.5 kg/cm² to 21.1 kg/cm² may still be used, as presses generating these types of pressures are readily available and comparatively inexpensive as compared to presses that are capable of generating about 1410 kg/cm² of pressure. For example, presses that generate between about 10.5 kg/cm² and 21.1 kg/cm² of pressure are routinely used in the wine and olive oil industries to press grapes and olives, respectively.

When sugarcane bagasse is used as the biomass from which the hydrolysate is pressed, fiber condition will generally be unimportant unless the biomass residue will be used as a feedstock for a paper mill. However, when the biomass residue derived from bagasse is used as a high energy density fuel replacement, the moisture content is an important factor. Therefore, higher, rather than lower pressures, may be desirable for purposes of performing the solid/liquid separation step 18. Similar considerations apply to solid/liquid separation step 28.

Pressing is also advantageous because it reduces dilution from wash water prior to solid/liquid separation. Wash water may be used to help separate the hydrolysate from the biomass. However, wash water will dilute the sugar stream and thus lower the resulting ethanol concentration in the fermented hydrolysate. If wash water is used, dilution of the sugar stream may be mitigated by the use of evaporators or similar machinery to reduce water content in the hydrolysate. The recovered water from evaporation may be recycled into subsequent wash processes. The addition of evaporation as a process step increases the sugar concentration of the hydrolysate and the ethanol concentration resulting from fermentation and thereby reduces the costs of distillation.

Once the sugar enriched hydrolysate including monosaccharides is separated from the biomass, there are a number of microbes that may be used for converting the monosaccharides of the sugar enriched hydrolysate into ethanol or other biofuels in fermentation step 24. For example, if the sugar enriched hydrolysate comprises a cellulose hydrolysate, so as to include glucose (which is a hexose), the glucose in the hydrolysate may be fermented by a number of yeast strains including Saccharomyces cerevisiae (traditional baker's yeast) and Kluyveromyces marxianus to name a few.

On the other hand, if the sugar enriched hydrolysate comprises a hemicellulose hydrolysate, the hydrolysate will include the pentoses xylose and arabinose, and a lower concentration of hexoses, except in the case of softwood hydrolysate. In the case of softwood hemicellulose, the hexose mannose is the major saccharide and the pentose xylose is the next most abundant. Microbes that can convert the combination of pentoses and hexoses found in hemicellulose hydrolysate into biofuels, such as ethanol, are not as abundant as those available for cellulose hydrolysate. To convert sugars from hemicellulose hydrolysate into ethanol, microbes that can ferment both five-carbon and six-carbon sugars are preferably utilized so that all of the available constituent sugars of the hemicellulose hydrolysate may be converted to ethanol or other biofuels. The same is true if the sugar enriched hydrolysate comprises a combination of cellulose hydrolysate and hemicellulose hydrolysate. Microbes that can ferment hexoses and pentoses may be derived from the genera Pachysolen, Kluyveromyces, Pichia, and Candida. Pachysolen tannophilus is preferably used in fermentation of a liquid hydrolysate comprising a hemicellulose hydrolysate. In particular, when immobilized, Pachysolen tannophilus has been found to effectively ferment hemicellulose hydrolysate produced from softwood.

In addition to immobilized yeasts, immobilized bacterium may also be used to ferment hexose and pentose sugars in sugar enriched hydrolysate. For example, the recombinant bacterium Zymomonas mobilis (NREL recombinant 8b) may be used to ferment hemicellulose hydrolysate produced from softwood, hardwood, and/or herbaceous sources.

Microbes with complementary metabolic properties may also be combined in the same fermentation process in step 24 to allow their complementary properties and abilities, such as complementary hexose and pentose fermentation capabilities or complimentary metabolic rates, to be used together. For example, because recombinant Zymomonas is unable to ferment mannose, the most prevalent sugar contained in softwood hydrolysate, the recombinant Zymomonas mobilis is preferably paired with a complementary yeast or bacterium that is able to effectively ferment the hexose mannose to ethanol or another biofuel when it used to ferment softwood hydrolysate. On the other hand, in the case of sugarcane bagasse, where the hydrolysate primarily comprises xylose and glucose, another microbe is not required to assist the recombinant Zymomonas to achieve a satisfactory fermentation of the contained sugars.

Other combinations of microbes are also possible including pairing different bacterium together, pairing different yeasts together, pairing various yeasts and bacterium together, or pairing or combining any number of microbes with complimentary features including using any number of microbes at the same time. As the number of combined microbes increases, however, their capabilities may begin to overlap significantly and thereby reduce the additive value of the additional microbes.

Although a portion of the free sugars in the biomass are prevented from degrading in pretreatment step 16 by being removed in solid/liquid separation step 18, some secondary products created from degradation during pretreatment 16 may still exist. In addition to secondary products created from degradation, other molecules may be extracted from the biomass by the pretreatment and/or saccharification conditions during the pretreatment and hydrolysis step 16. These extracted compounds may include terpenes, sterols, fatty acids, and resin acids. These extracted compounds can also be inhibitory to metabolic processes, including fermentation, in yeast and other microbes, such as bacteria.

Furthermore, metal cations including calcium, aluminum, potassium, and sodium may be found in hydrolysate and heavy metals may be present from degradation of the metal vessels due to hydrolysis. The presence of such metal cations may also be inhibitory above certain concentrations.

In one embodiment of the method as shown in FIG. 3, the biomass residue (labeled solids in FIG. 3) that is the product of solid/liquid separation step 18, may be combusted as fuel for the sugar mill plant in an optional combustion step 23. An additional benefit of the processes of the present patent document is that the additional washing and pressing steps may further remove soluble cations and inorganic elements from the biomass residue prior to combustion 23. Furthermore, the use of hydrolysate in the elution may enhance the elution of the soluble cations over the use of just water because in an acid solution the cations solubility is increased.

Removing cations and inorganic elements from the biomass residue is important because such biomass residue is often burned as fuel in combustion chambers. During combustion, the cations and inorganic elements are released from the biomass and deposit on the surfaces of the combustion unit. This is called fouling. Slagging relates to the melting of these deposits, forming a glassy layer. Interaction of the deposits with the metal surfaces can accelerate corrosion, which gradually destroys the metal surface, leading to increased maintenance requirements and reduced service life of the installation.

Current methods of dealing with slagging often involve adding mineral additives, mainly lime-/dolimite based additives and/or clay or kaolinite based additives, to combat ash related operational problems such as fouling, slagging, and corrosion. However, the methods and processes of the present patent document may reduce fouling, slagging and the resulting corrosion by removing some the soluble cations and inorganic elements that cause fouling, slagging and corrosion prior to combustion 23.

As may be seen by comparing FIG. 2 and FIG. 3 to FIG. 1, both FIG. 2 and FIG. 3 contain and additional mixing step 14 and an additional solid/liquid separation step 28 prior to pretreatment 16. The addition of the mixing step 14 and solid/liquid separation step 28 will reduce the soluble cations in the biomass residue prior to combustion 23, similar to the way the additional steps reduce the free sugars. Accordingly, if the solids from solid/liquid separation step 18 are combusted as fuel, they may exhibit less fouling, slagging and corrosion of the combustion chamber.

If the solid/liquid separation steps are done by pressing, a large percentage of the moisture may be removed from the biomass residue. The more moisture that is removed from the biomass residue, the greater the reduction in the concentration of slag forming constituents present in the biomass residue. By repetitive mixing and pressing, the concentration of slag forming constituents may be further reduced. This is because when the biomass is washed or mixed with hydrolysate or other solutions, the slag forming constituents may be eluted into the solution similar to the free sugars. This allows the subsequent separation steps, preferably done by pressing, to reduce the concentration of slag forming constituents. Additional mixing and pressing steps other than those shown in the embodiments of the figures may be used. In addition, the amount of soluble cations and other inorganic material that will be removed from the biomass residue will be substantially increased if the solid/liquid separation step is performed using a press to reduce the moisture content of the biomass, preferably down to 50% or less by weight, more preferably down to 30% or less.

In one embodiment of the method, a substantial amount of the soluble cations that cause fouling are removed from the biomass residue. In a preferred embodiment, a majority of the soluble cations that cause fouling are removed from the biomass residue (solids) prior to combustion 23. In other embodiments, the concentration of slag forming constituents of the biomass residue, including the soluble cations, is substantially reduced prior to combustion. By non-limiting example slag forming constituents may include but are not limited to cations of Silicon (Si), Potassium (K), Calcium (Ca), Magnesium (Mg), and other inorganic elements found in biomass.

In order to deal with the potential for high levels of inhibitory secondary products often found in biomass hydrolysate—for example, levels that would inhibit the fermentation microbes in their free state—during the fermentation step 24, the fermentation microbes may be immobilized, and more preferably immobilized in calcium alginate. Immobilization confers an increased resistance of microbes to inhibitors and therefore, increases fermentation efficiency. For example, immobilization in calcium alginate greatly reduces the susceptibility of the yeast Pachysolen tannophilus to inhibitors contained in softwood hydrolysate. Preferably the calcium alginate, or other material used to immobilize the microbes, is in a form with a high surface area such as in a bead, sponge, or mesh form.

By immobilizing the fermentative microbe(s) during the fermentation step 24, the need for conditioning 22 the biomass hydrolysate to reduce the concentration of, or possibly even completely remove, inhibitory secondary products is significantly ameliorated. This is because the need to lower the concentration of inhibitory secondary products to the levels necessary for fermentation using free microbes is eliminated. Thus, as reflected in FIGS. 2 and 3, conditioning step 22 to reduce the concentration of inhibitors is an optional step.

Conditioning the biomass hydrolysate in conditioning step 22 to reduce the concentration of inhibitory secondary products may still be desirable where, for example, the concentration of the secondary products (either individually or in combination) is sufficiently high to interfere with the fermentation of sugars even by the immobilized microbe(s). In such cases, however, the concentration of the inhibitory secondary products will generally not need to be reduced to the same levels as necessary for fermentation using free microbes and thus a less severe and less costly conditioning process may be employed. To offset the costs associated with the overall fermentation process, it may also be desirable to recover secondary products having a high value through an optional high value secondary product recovery step.

In some instances, it may also be desirable to perform conditioning step 22 even when the concentration of inhibitory secondary products is insufficient to inhibit fermentation by the immobilized microbe(s) where, for example, the secondary products have high value and thus it is desirable to separately recover the high value secondary products. This may be desirable, for example, where the net value of the recovered high value secondary products may be used to offset, and hence lower, the costs associated with the overall fermentation process. Conditioning step 22 may also be used to raise the sugar levels prior to fermentation by removing water from the hydrolysate.

There are numerous methods of performing the conditioning step 22 to reduce the concentrations of inhibitory secondary products. Employing different conditioning methods for conditioning step 22 will result in different concentration levels of inhibitory secondary products remaining in the hydrolysate. The method of conditioning chosen for conditioning step 22 may depend on a variety of factors, including the sensitivity of the microbe used during fermentation to inhibitory secondary products, costs, and whether there is a desire to recover high value secondary products. The more sensitive the microbe, the more desirable it will be to reduce the concentration of the inhibitory products from the biomass hydrolysate during conditioning of the hydrolysate in step 22. Immobilization of the fermentative microbe(s), however, will decrease the sensitivity of the microbe to inhibitory secondary products and thus may reduce the complexity and costs incurred during conditioning step 22. Some of the conditioning methods that may be employed in conditioning step 22 to reduce the concentration of secondary products include, but are not limited to: 1) overliming of hydrolysate; 2) activated carbon (AC) treatment followed by pH adjustment; 3) ion exchange followed by overliming; 4) AC treatment followed by ion exchange; and 5) AC treatment followed by membrane filtration.

During the conditioning process 22, the inhibitory secondary products, which have value when isolated, may be recovered. Hydrolysate from solid/liquid separation 18 contains a number of high value secondary products including, but not limited to the mineral acid used in the pretreatment process 16, such as sulfuric acid, acetic acid hydrolyzed from hemicellulose polymers, anti-oxidant molecules (phenolic and polyphenolic compounds) liberated from the partial hydrolysis of lignin, other organic acids, nutraceutical, cosmeceutical, or pharmaceutical products, and different furans and furan derivatives, such as 5-hydroxymethylfurfural and furfural. High value secondary product recovery may be accomplished by adsorption to different matrices, including activated carbon, ion exchange resin, ion exchange membrane, organic molecule “scavenging” resins, polystyrene beads, or another such medium with a high surface area. High value secondary product recovery may also be accomplished by separating them from soluble hexoses and pentoses through ion exclusion chromatography, pseudo-moving bed chromatography, high performance liquid chromatography or by filtration methods including micro-, nano-, and ultrafiltration using hollow fiber or other membrane technologies. High value secondary product recovery may include several of the aforementioned processes in series to recover different molecular species. Furthermore, the recovery processes may be tailored to recover specific secondary products according to the nature of the starting biomass. Recovery of high value secondary products also provides a benefit to the fermentation process 24, as many of the recovered secondary products (acetic acid, furans and their derivatives, phenolic and polyphenolic compounds, levulinic acid, formic acid, and others) are inhibitory to yeast and bacterial fermentation of sugars to ethanol. Thus, recovery of high value secondary products both increases the economics of the entire process and allows for more efficient fermentation 24 of the pentoses and hexoses.

The optional recovery of high value products from the hydrolysate may occur at different times during the processes exemplified in FIG. 2 and FIG. 3. For example, the high value products may be recovered anytime after the pretreatment step 16. In addition, because the hydrolysate is recycled back into mixing step 14, the recovery of high value products may occur after the solid/liquid separation step 28 but prior to fermentation and distillation.

Even following partial recovery of many high value products, the concentrations of these products may remain elevated, and considering the synergistic nature of the inhibitors, are sufficient to interfere with fermentation 24 of sugars to ethanol. In order to more efficiently ferment the hexoses and pentoses that have been separated from the biomass residue and high value products, the fermentation microbes may be immobilized. Immobilization confers an increased resistance of microbes to inhibitors and therefore, increases fermentation efficiency. For example, immobilization in a calcium alginate greatly reduces the susceptibility of microbes, such as the yeast Pachysolen tannophilus, to inhibitors contained in softwood hydrolysate. Preferably the calcium alginate, or other material used to immobilize the microbes, is in a form with a high surface area such as in bead, sponge, or mesh form.

A variety of bioreactor designs, including a traditional non-stirred fermenter or stirred fermenter, may be used for the fermentation of the biomass hydrolysate using free or immobilized microbes. The reactor may be a submerged reactor or other type of liquid reactor. In order to provide the highest yield, a submerged reactor is preferable to ferment five-carbon sugars.

In the case of microbes that are immobilized, a packed bed reactor could be utilized, or a tankage system similar to that employed for carbon-in-pulp processes in the gold mining industry could be used. In the latter, beads would be moved counter-current to the solution flow and could be easily recovered for regeneration. Thin film reactors may also work well with immobilized microbes.

In addition, solid/liquid contactors may be used with immobilized microbes. These types of reactors include ion exchange columns, packed bed reactors, trickle flow reactors, and rotating contactors. Other reactors that may be used are fluidized-bed and upflow type reactors.

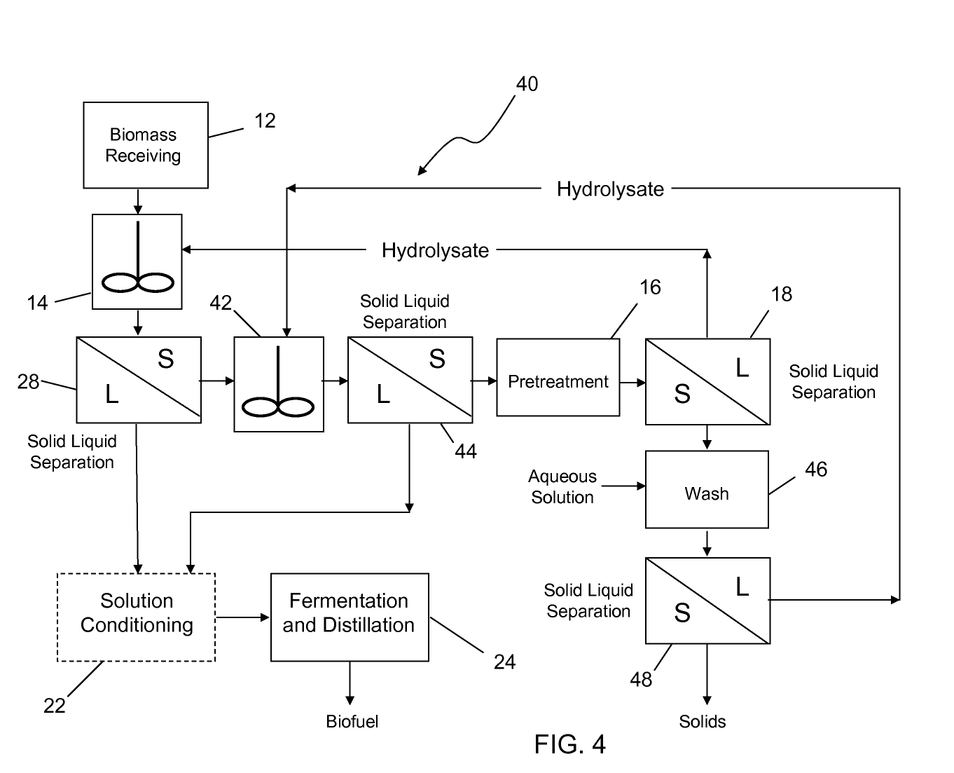

FIG. 4 illustrates an embodiment of a process 40 for producing biofuel from biomass that includes free sugars. The process 40 is similar to the process 20 shown in FIG. 2 except additional process steps 42, 44, 46, and 48 have been added and a second hydrolysate is recycled.

In process 40 shown in FIG. 4, the biomass solids that result from the solid/liquid separation step 28 enter an additional mixing step 42 and subsequent additional solid/liquid separation step 44 prior to entering the pretreatment step 16. The additional mixing step 42 and solid/liquid separation step 44 help remove even more of the free sugars present in the biomass prior to entering pretreatment 16.

As may be seen in FIG. 4, in some embodiments the aqueous solution used for the additional mixing step 42 may be recycled from the additional solid/liquid separation step 48. Prior to the additional solid/liquid separation step 48, an additional wash step 46 is used to combine the biomass residue solids from the solid/liquid separation step 18 with an aqueous solution to further elute any monosaccharides that were produced during pretreatment and/or hydrolysis 16. The mixture produced during the wash step 46 includes a diluted hydrolysate. The mixture produced during wash step 46 then goes through an additional solid/liquid separation step 48. A diluted hydrolysate is separated from the biomass residue solids. The diluted hydrolysate contains additional remaining monosaccharides. The diluted hydrolysate is recycled as the liquid solvent for the mixing step 42.

Although the embodiments shown in FIGS. 2 and 3 show a single solid/liquid separation step before and after pretreatment, and FIG. 4 illustrates an embodiment with two (2) solid/liquid separation steps before and after pretreatment 16, other embodiments may have any number of solid/liquid separation steps and/or recycled solutions.

An exemplary embodiment of the process shown in FIG. 4 will now be described with respect to a sugar mill. In the example, numerous percentages, ratios, and amounts will be discussed. These values are not meant to be absolute but only representative of one exemplary embodiment. Other percentages, ratios and amounts may occur in other embodiments.

For example, the exemplary embodiment describes a sugar mill that produces both sugar and ethanol. In the exemplary embodiment, sugarcane enters the sugar mill with about 15% sucrose (free sugars) by weight. A sugar mill will typically extract around 14% of the 15% sucrose into sugar syrups of various concentrations and the 1% balance remains with the bagasse. Many modern sugar mills that also produce ethanol split the sugar syrups equally for sugar and ethanol production. The present patent document teaches methods and apparatuses for also extracting a substantial amount of the 1% balance that remains with the bagasse for further processing into ethanol.

While modern sugar/ethanol mills often split the sugar syrups equally for sugar and ethanol production, the ratio of free sugar used to make consumable sugar versus the percentage of free sugar that is used for ethanol production may be different than 1:1 in embodiments of the present patent document. For example the ratio may be 3:1 or 2:1 or even 1:3 or 1:2 or any other ratio. In particular, the sugar mill may change the ratio of free sugar used to create consumable sugar and ethanol based on the market demand. For example, as the price of ethanol goes up and the price of sugar goes down the plant might shift the ratio more towards ethanol. As another example, the sugar mill may begin to have excess inventory of either sugar or ethanol and want to shift the ratio towards the other product to allow the overstocked inventory to be decreased. Similar to the ratio of sugar used for the sugar and ethanol production, the ratios given below in this exemplary embodiment are given with respect to the exemplary embodiment described and other ratios, percentages, and values may be used in other embodiments.

One (1) tonne of fresh wet sugarcane (Labeled A in Table 1) enters a sugar mill. It may be in any solid to liquid ratio, however, for this example, the wet sugarcane is approximately 88% liquid and 12% solid. The wet sugarcane contains approximately 15% sucrose (15% free sugars) when it enters the sugar mill. The sugarcane is processed by the sugar mill into a recovered sugar solution and residue bagasse (Labeled B and C respectively in Table 1). In this exemplary embodiment, the sugarcane is processed by the sugar mill and 14% of the free sugars are removed for processing into sugar/ethanol and the bagasse is sent for further sugar extraction for processing into ethanol. As explained above, these ratios may be varied on purpose or because of other factors. However, 14% free sugar removal with a 1% sugar remainder in the bagasse is a typical value for sugar mills that are not processing the sugarcane with any special treatment to try and extract additional free sugars.

During the processing of the sugarcane by the sugar mill, which may be done by pressing or other means, a substantial amount of the moisture is removed and the 1 tonne of wet sugarcane is reduced to approximately 0.24 tonne of bagasse with a moisture content of 50% (0.12 tonne solid and 0.12 tonne liquid). Because a substantial amount of the mass of the sugarcane is extracted with the 14% free sugar removal, the overall sugar content of the remaining bagasse is about 4.2%. Table 1 depicts the ratios and percentages.

TABLE 1

| | | Sucrose (free | | | Sucrose (free | Sugars | |
|---|---|---|---|---|---|---|---|
| | | sugar) content | Moisture | Solid | sugars) in | (grams/ | Total Mass |
| | | % of 1 Tonne | Content | Content | Tonne (t) | Liter) | in Tonne (t) |
| A | Wet Sugarcane | 15% | 88% | 12% | 0.15 | 170.5 | 1.0 |
| B | Recovered Sugar | 14% | 100% | 0% | 0.14 | 184.2 | 0.76 |
| C | Bagasse | 1% | 50% | 50% | 0.01 | 83.3 | .24 |

The bagasse (C), which contains 0.1 tonne or 83.3 grams/liter (g/l) of free sugars at this stage, enters the mixing process 14 where it is mixed with an aqueous solution such as hydrolysate from a solid/liquid separation step 18 (Labeled D in Table 2). In this exemplary embodiment, the location of the solid/liquid separation step 18 where the aqueous solution is recycled from is after the pretreatment step 16. Because the aqueous solution is recycled from after the pretreatment step 16, the aqueous solution may be a hydrolysate. In this exemplary example, for purposes of illustration of one embodiment of the process, about 0.32 tonne of hydrolysate is recycled to mixing step 14. The 0.32 tonne of hydrolysate contains 0.016 tonne of sucrose (free sugars) at about 50 g/l.

In a preferred embodiment, the bagasse (C) and hydrolysate (D) are mixed for a sufficient time to allow a majority of the free sugars in the bagasse to be eluted into the hydrolysate. Once the hydrolysate (D) is mixed with the incoming bagasse (C) for a sufficient time, a bagasse solution (C+D) is passed to the solid liquid separation step 28. Although for this exemplary example (C+D) results in a bagasse solution that is about 79% liquid, other solid to liquid ratios are possible. In a preferred embodiment, the solid to liquid ratio of the bagasse solution (C+D) has a range of 60 to 85% liquid.

The solid/liquid separation step 28 separates the bagasse solution (C+D) into a liquid pressate (Labeled E in Table 2) which includes sugar enriched hydrolysate, and a bagasse solution (F). For purposes of this exemplary example, the solid/liquid separation step reduces the liquid content of the bagasse solution (F) to 30% liquid and 70% solid. In other embodiments, the pressing step 28 may result in more or less liquid being separated from the solid bagasse resulting in a different solid to liquid ratio for bagasse solution (F). For example, the pressing step 28 may extract more liquid and the resulting bagasse solution (F) may be 25% liquid and 75% solid. Other solid to liquid ratios of bagasse solution (F) may exist in other embodiments including 50% or 55% liquid and 50% or 45% solid respectively. The more liquid that is separated from the solid bagasse the more free sugars that may be extracted. For this embodiment, the properties of both the pressate (E) and the bagasse solution (F) after solid liquid separation step 28 are shown in Table 2.

TABLE 2

| | | | | Sucrose (free | Sugars | |
|---|---|---|---|---|---|---|
| | | Moisture | Solid | sugars) in | (grams/ | Total Mass |
| | | Content | Content | Tonne (t) | Liter) | in Tonne (t) |
| D | Hydrolysate 1 | 100% | 0% | 0.016 | 50.0 | 0.32 |
| C + D | Bagasse Solution entering | 79% | 21% | 0.026 | 59.1 | 0.56 |
| | S/L separation step 28. | | | | | |
| E | Pressate after S/L | 100% | 0% | 0.023 | 59.1 | 0.389 |
| | separation step 28. | | | | | |
| F | Solid after S/L separation | 30% | 70% | 0.003 | 59.1 | 0.171 |
| | step 28. | | | | | |

The bagasse solution (F) is next passed into a second mixing step 42 where it is mixed with a second aqueous solution (Labeled G in Table 3). Similar to the mixing step 14, the aqueous solution (G) may be hydrolysate or a diluted hydrolysate from a process step after pretreatment step 16 such as recycling the diluted hydrolysate from solid/liquid separation step 48. The combination of the bagasse solution (F) and diluted hydrolysate (G) is then passed into another solid liquid separation step 44 where it is separated into a pressate (H) (which includes sugar enriched hydrolysate) containing 0.004 tonne of free sugar and a bagasse solution (J) with 0.001 tonne of free sugars.

TABLE 3

| | | | | Sucrose (free | Sugars | |
|---|---|---|---|---|---|---|
| | | Moisture | Solid | sugars) in | (grams/ | Total Mass |
| | | Content | Content | Tonne (t) | Liter) | in Tonne (t) |
| G | Hydrolysate 2 | 100% | 0% | 0.002 | 4.4 | 0.4 |
| F + G | Bagasse Solution entering | 79% | 21% | 0.005 | 10.6 | 0.571 |
| | S/L separation step 44. | | | | | |
| H | Pressate after S/L | 100% | 0% | 0.004 | 10.6 | 0.4 |
| | separation step 44. | | | | | |
| J | Solid after S/L separation | 30% | 70% | 0.001 | 10.6 | 0.171 |
| | step 44. | | | | | |

At this point over 99% of the free sugars that were in the original bagasse (C) that entered the process 40 have been extracted. The bagasse solution (J) only contains 0.001 tonne of free sugars. Accordingly, the bagasse (J) may be further processed in pretreatment step 16 to release some of the sugars bound up in the cellulose and hemicellulose for further extraction. Depending on the pretreatment process used, water, steam and/or acid may all be added to the bagasse solution (J) during pretreatment 16. The result of the pretreatment step 16 is a hydrolysate solution (K) that includes a biomass residue (L). The properties of the resulting hydrolysate solution (K) are shown in Table 4. As may be seen in Table 4, 0.018 tonne of sugar (K-J) is extracted during pretreatment step 16 from the 0.171 tonne of bagasse residue (0.12 tonne of solid bagasse and 0.051 tonne of liquid).

The hydrolysate solution (K) is then put through a solid liquid separation step 18. The solid liquid separation step 18 separates the hydrolysate solution (K) into a hydrolysate (D), which is recycled as explained above, and a biomass residue (L). The hydrolysate (D) from solid/liquid separation step 18 is recycled back into mixing step 14. The biomass residue (L) continues on to the wash step 46.

TABLE 4

| | | | | Sucrose (free | Sugars | |
|---|---|---|---|---|---|---|
| | | Moisture | Solid | sugars) in | (grams/ | Total Mass |
| | | Content | Content | Tonne (t) | Liter) | in Tonne (t) |
| K | Hydrolysate | 80% | 20% | 0.019 | 50.0 | 0.45 |
| L | Biomass residue after S/L | 30% | 70% | 0.002 | 50.0 | 0.13 |
| | separation step 18. | | | | | |

In wash step 46, the biomass residue may be washed with an aqueous solution. The aqueous solution may be any combination of reagents designed to help extract the remaining sugars. In this exemplary example, the aqueous solution is 0.4 tonne of water (M).

TABLE 5

| | | | | Sucrose (free | Sugars | |
|---|---|---|---|---|---|---|
| | | Moisture | Solid | sugars) in | (grams/ | Total Mass |
| | | Content | Content | Tonne (t) | Liter) | in Tonne (t) |
| M | Aqueous Solution | 100% | 0% | 0.000 | 0.0 | 0.4 |
| L + M | Biomass Residue Solution | 83% | 17% | 0.002 | 4.4 | 0.53 |
| | entering S/L separation | | | | | |
| | step 48. | | | | | |
| N | Solid after S/L separation | 30% | 70% | 0.000 | 4.4 | 0.13 |
| | step 48. | | | | | |

The biomass residue solution (L+M) that is formed after the aqueous solution (M) is added to the biomass residue (L), is then passed to solid/liquid separation step 48. Solid/liquid separation step 28, separates the biomass residue solution (L+M) into a diluted hydrolysate (G) and a washed biomass residue (N). As may be seen from Table 5, the washed biomass residue (N) has virtually no remaining free sugars. The washed biomass residue (N) may be discarded, used as fuel for the plant, or turned into some other high value product. The diluted hydrolysate (G) is recycled back to mixing step 42.

The above discussion tracked the solids path (process steps 12, 14, 28, 42, 44, 16, 18, 46 and 48) through an exemplary embodiment of the process 40. However, the liquid (pressate E and H a.k.a. sugar enriched hydrolysate and sugar enriched diluted hydrolysate), from solids/liquids steps 28 and 44 respectively, is further processed into a biofuel. The combination of pressate (E+H) is shown in Table 6.

TABLE 6

| | | | | Sucrose (free | Sugars | |
|---|---|---|---|---|---|---|
| | | Moisture | Solid | sugars) in | (grams/ | Total Mass |
| | | Content | Content | Tonne (t) | Liter) | in Tonne (t) |
| E + H | Final Pressate to | 100% | 0% | 0.0272 | 34.5 | 0.79 |
| | Fermentation | | | | | |

The final pressate (E+H) may be conditioned in solution conditioning step 22 as discussed above. However, the solution conditioning step 22 is optional and the final pressate (E+H) may proceed directly to fermentation and distillation. Given an Ethanol yield of 588.9 liters per tonne of sugar or an 86.6% yield, the 0.12 tonne of bagasse would yield 15.99 liters (4.23 gallons) of ethanol. This results in a yield of 35.26 gallons of ethanol per tonne of bagasse.

The above example extracts 0.0175 tonne of extra sugars from the solid bagasse during the pretreatment process. Subtracting this amount from the sucrose of (E+H) leaves 0.0097 tonne of sugar. This is the amount of the free sugar that was extracted from the original 0.01 tonne of free sugar in the bagasse (C). This results in an extraction of 97% of the free sugar originally found in the wet bagasse residue. To this end, the free sugars in the bagasse are converted into 5.7 liters of ethanol or 1.5 gallons which results in a yield of 12.5 gallons of ethanol per tonne of dry bagasse from just the free sugars not extracted during the original sugar mill processing.

Although the exemplary example discussed herein resulted in 97% of the free sugar originally found in the bagasse being extracted, other percentages may result. In the preferred embodiments, approximately 80% to 99% of the free sugar in the bagasse is extracted for conversion into biofuel.

Although the embodiments have been described with reference to the drawings and specific examples, it will readily be appreciated by those skilled in the art that many modifications and adaptations of the processes and apparatuses described herein are possible without departure from the spirit and scope of the embodiments as claimed herein. Thus, it is to be clearly understood that this description is made only by way of example and not as a limitation on the scope of the embodiments as claimed below.

What is claimed is:

1. A process for producing biofuel from biomass that includes free monosaccharides comprising the steps of:
mixing the biomass with a recycled hydrolysate for a sufficient time to elute a portion of the free monosaccharides from the biomass thereby forming a sugar enriched hydrolysate;
separating sugar enriched hydrolysate from the biomass; and
fermenting monosaccharides in the separated sugar enriched hydrolysate.

2. The process of claim 1, wherein the mixing step is performed for a time sufficient to elute a majority of the free monosaccharides into the recycled hydrolysate.

3. The process of claim 2, wherein 90% or more of the free monosaccharides are eluted into the recycled hydrolysate.

4. The process of claim 1, further comprising the step of pretreating the biomass after the separating step to produce hydrolysate and a biomass residue.

5. The process of claim 4, further comprising the step of separating hydrolysate from the biomass residue.

6. The process of claim 5, wherein the separated hydrolysate is used as the recycled hydrolysate in the mixing step for a new batch of biomass that contains free monosaccharides.

7. The process of claim 1, wherein the biomass is sugarcane bagasse.

8. The process of claim 4, wherein the biomass residue is converted into a high value product.

9. The process of claim 7, wherein the sugarcane bagasse is a byproduct from a sugar mill.

10. The process of claim 1, wherein the separating step is performed by pressing.

11. The process of claim 5, wherein a concentration of slag forming constituents are substantially reduced in the biomass residue.

12. The process of claim 1, wherein the monosaccharides are fermented using an immobilized microbe.

13. A process for producing biofuel from biomass that includes free sugars comprising the steps of:
mixing the biomass with a liquid solution until a substantial percentage of free sugars in the biomass are eluted into the liquid solution, wherein the biomass is mixed with the liquid solution prior to pretreating the biomass;
separating liquid solution including free sugars from the biomass; and
fermenting free sugars in the separated liquid solution.

14. The process of claim 13, wherein the liquid solution is hydrolysate.

15. The process of claim 14, further comprising the step of pretreating the biomass after the separating step to produce hydrolysate and a biomass residue.

16. The process of claim 15, further comprising the step of separating the biomass residue from the hydrolysate.

17. The process of claim 15, further comprising the step of creating a high value product from the biomass residue.

18. The process of claim 13, wherein the biomass is sugarcane bagasse.

19. The process of claim 18, wherein the sugarcane bagasse is a byproduct of a sugar mill.

20. The process of claim 16, wherein the separated hydrolysate is used as the liquid solution in the mixing step for a new batch of biomass that includes free sugars.

21. A process for producing biofuel from biomass that includes free sugars comprising the steps of:
mixing the biomass with a hydrolysate prior to pretreating the biomass;
allowing free sugars contained within the biomass to be eluted by the hydrolysate;
separating hydrolysate including the eluted free sugars from the biomass; and
fermenting free sugars contained in the separated hydrolysate.

22. The process of claim 21, further comprising the step of pretreating the biomass to produce a mixture of hydrolysate and biomass residue.

23. The process of claim 22, further comprising the step of separating hydrolysate from the mixture.

24. The process of claim 21, wherein the biomass is sugarcane bagasse.

25. The process of claim 24, wherein the sugarcane bagasse is a byproduct of a sugar mill.

26. The process of claim 22, further comprising the step of producing a high value product from the biomass residue.

27. The process of claim 23, wherein the separating step is performed by pressing.

28. The process of claim 27, wherein a concentration of slag forming constituents are substantially reduced in the biomass residue.

29. The process of claim 21, wherein the free sugars are fermented using an immobilized microbe.
